(19) United States
(12) Patent Application Publication
(10) Pub. No.: US 2025/0117952 A1
(43) Pub. Date: Apr. 10, 2025

(54) AUTOMATED SIDE BRANCH DETECTION AND ANGIOGRAPHIC IMAGE CO-REGISRATION

(71) Applicant: Boston Scientific Scimed, Inc., Maple Grove, MN (US)

(72) Inventors: Hatice Cinar Akakin, Eden Prairie, MN (US); Kevin Bloms, Minneapolis, MN (US); Wenguang Li, Los Gatos, CA (US)

(73) Assignee: Boston Scientific Scimed, Inc., Maple Grove, MN (US)

(21) Appl. No.: 18/903,068

(22) Filed: Oct. 1, 2024

Related U.S. Application Data
(60) Provisional application No. 63/588,552, filed on Oct. 6, 2023.

(51) Int. Cl. G06T 7/30; G06T 7/00; G06T 7/10

(52) U.S. Cl. CPC G06T 7/30; G06T 7/0012; G06T 7/10; G06T 2207/10081; G06T 2207/10101; G06T 2207/10132; G06T 2207/30101

(57) ABSTRACT

The present disclosure provides to generate a side branch mask from an extravascular image of a vessel using an ensemble of machine learning (ML) models. The side branch mask can be generated by inferring, using several initial ML models, indications of side branches from an image frame and inferring, using a post-processing ML model, the side branch mask from the indication of side branches.

### CROSS-REFERENCE TO RELATED APPLICATIONS

This application claims the benefit of U.S. Provisional Patent Application Ser. No. 63/588,552 filed on Oct. 6, 2023, the disclosure of which is incorporated herein by reference.

### TECHNICAL FIELD

The present disclosure pertains to coronary angiography and to co-registration of coronary angiograms with intravascular and extravascular imaging modalities.

### BACKGROUND

Coronary angiography (angiogram) or CT coronary angiography (CTA or CCTA) is the use of X-ray imaging modalities to assess the coronary arteries of the heart. For example, a patient receives an intravenous injection of contrast agent and then the heart is scanned using a high speed CT scanner. CTA is often used in conjunction with other imaging modalities, such as, intravascular ultrasound (IVUS) or intravascular optical CT (OCT). A physician will use the X-ray imaging modality and the IVUS or intravascular OCT images to assess the extent of an occlusion (or occlusions) in the coronary arteries, usually to diagnose coronary artery disease.

To aid physicians in reviewing these images, they can be co-registered to each other. For example, each image in a series of IVUS images can be mapped, or co-located, to a position of the vessel represented in the X-ray image. Co-registration is often facilitated by first identifying fiducials (e.g., side branches, or the like) in the images and then mapping these fiducials to each other. Accordingly, there is a need to identify fiducials in each type of image and map these fiducials to each other.

### BRIEF SUMMARY

The present disclosure provides to identify side branch locations on a coronary angiography image in an automated manner (e.g., without user input or with limited user input). The disclosure provides methods and systems to identify a mask of side branch locations from the coronary angiography image or from the coronary angiography image and intravascular (e.g., IVUS, or the like) images.

In some embodiments, the disclosure can be implemented as a method for a vascular co-registration system. The method can comprise receiving, at a processor of a vascular co-registration system, an image frame associated with a vessel of a patient; inferring, by the processor using a plurality of initial machine learning (ML) models, indications of at least one side branch of the vessel from the image frame; and inferring, by the processor using a post-processing model, a side branch mask from the indications of the at least one side branch of the vessel, wherein the image frame is captured using a first imaging modality, and wherein the image frame can be co-registered to one or more other image frames based on the side branch mask, wherein the one or more other modality image frames are captured using a second imaging modality different than the first imaging modality.

With further embodiments of the method, the first imaging modality is angiography and wherein the second imaging modality is intravascular ultrasound or optical coherence tomography or CT angiography.

With further embodiments, the method can comprise co-registering, by the processor, the first image frame with the one or more other image frames based in part on the side branch mask.

With further embodiments, the method can comprise generating, by the processor, a graphical information element comprising indications of the image frame and the side branch mask and sending the graphical information element to a display.

With further embodiments of the method, inferring indications of the at least one side branch of the vessel from the image frame comprises inferring, by the processor, indications of the at least one side branch of the vessel from the image frame and a vessel centerline.

With further embodiments, the method can comprise receiving, by the processor, the vessel centerline.

With further embodiments, the method can comprise determining, by the processor, the vessel centerline.

With further embodiments of the method, the side branch mask comprises an indication of locations of the one or more side branches with respect to the centerline of the vessel.

With further embodiments of the method, inferring indications of the at least one side branch of the vessel from the image frame comprises inferring indications of the at least one side branch of the vessel from the image frame and a segmentation of the image frame.

With further embodiments, the method can comprise receiving, by the processor, the segmentation of the image frame or determine the segmentation of the image frame.

With further embodiments, the method can comprise receiving, by the processor, a plurality of intravascular images of the vessel; and inferring, by the processor, indications of the at least one side branch of the vessel from the image frame and the plurality of intravascular image frames.

With further embodiments of the method, the plurality of initial ML models comprising a first group ML models and a second group of ML models, wherein the first group of ML models is different than the second group of ML models, and wherein inferring indications of the at least one side branch of the vessel from the image frame and the plurality of intravascular image frames comprises inferring, by the processor using the first group of ML models, ones of the indications of the at least one side branch of the vessel from the image frame; and inferring, by the processor using the second group of ML models, other ones of the indications of the at least one side branch of the vessel from the plurality of intravascular images.

With further embodiments of the method, the side branch mask is a one-dimensional (1D) mask defined with respect to the image frame, wherein the image frame is one of a plurality of image frames in a cine loop, and/or wherein the image frame is a 256 pixel by 256 pixel angiography image.

In some embodiments, the disclosure can be implemented as a computer-readable storage device, comprising instructions executable by a processor of a computing device coupled to an intravascular imaging device and a fluoroscope device, wherein when executed the instructions cause the computing device to implement any of the methods disclosed herein.

In some embodiments, the disclosure can be implemented as an apparatus comprising a processor arranged to be coupled to an intravascular imaging device and/or a fluoroscope device, the apparatus further comprising a memory comprising instructions, the processor arranged to execute the instructions to implement any of the methods disclosed herein.

In some embodiments, the disclosure can be implemented as an apparatus for viewing images of a vessel and for identifying side branches from an image. The apparatus can comprise a processor and a memory storage device coupled to the processor, the memory comprising instructions executable by the processor, the instructions when executed cause the apparatus to receive an image frame associated with a vessel of a patient; infer, using a plurality of initial machine learning (ML) models, indications of at least one side branch of the vessel from the image frame; and infer, using a post-processing model, a side branch mask from the indications of the at least one side branch of the vessel, wherein the image frame is captured using a first imaging modality, and wherein the image frame can be co-registered to one or more other image frames based on the side branch mask, wherein the one or more other modality image frames are captured using a second imaging modality different than the first imaging modality.

With further embodiments of the apparatus, the first imaging modality is angiography and wherein the second imaging modality is intravascular ultrasound or optical coherence tomography or CT angiography.

With further embodiments of the apparatus, the instructions when executed further cause the apparatus to co-register the first image frame with the one or more other image frames based in part on the side branch mask.

With further embodiments of the apparatus, the instructions when executed further cause the apparatus to generate a graphical information element comprising indications of the image frame and the side branch mask and send the graphical information element to a display.

With further embodiments of the apparatus, the instructions when executed to infer indications of the at least one side branch of the vessel from the image frame cause the apparatus to infer indications of the at least one side branch of the vessel from the image frame and a vessel centerline.

With further embodiments of the apparatus, the instructions when executed further cause the apparatus to receive the vessel centerline.

With further embodiments of the apparatus, the instructions when executed further cause the apparatus to determine the vessel centerline.

With further embodiments of the apparatus, the side branch mask comprises an indication of locations of the one or more side branches with respect to the centerline of the vessel.

With further embodiments of the apparatus, the instructions when executed to infer indications of the at least one side branch of the vessel from the image frame cause the apparatus to infer indications of the at least one side branch of the vessel from the image frame and a segmentation of the image frame.

With further embodiments of the apparatus, the instructions when executed further cause the apparatus to receive the segmentation of the image frame or determine the segmentation of the image frame.

With further embodiments of the apparatus, the instructions when executed further cause the apparatus to receive a plurality of intravascular images of the vessel; and infer indications of the at least one side branch of the vessel from the image frame and the plurality of intravascular image frames.

With further embodiments of the apparatus, the plurality of initial ML models comprising a first group ML models and a second group of ML models, wherein the first group of ML models is different than the second group of ML models, and the instructions when executed to infer indications of the at least one side branch of the vessel from the image frame and the plurality of intravascular image frames cause the apparatus to infer, using the first group of ML models, ones of the indications of the at least one side branch of the vessel from the image frame; and infer, using the second group of ML models, other ones of the indications of the at least one side branch of the vessel from the plurality of intravascular images.

With further embodiments of the apparatus, the side branch mask is a one-dimensional (1D) mask defined with respect to the image frame, wherein the image frame is one of a plurality of image frames in a cine loop, and/or wherein the image frame is a 256 pixel by 256 pixel angiography image.

In some embodiments, the disclosure can be implemented as a computer-readable storage device, comprising instructions executable by a processor of a computing device coupled to an intravascular imaging device and/or a fluoroscope device, wherein when executed the instructions cause the computing device to receive an image frame associated with a vessel of a patient; infer, using a plurality of initial machine learning (ML) models, indications of at least one side branch of the vessel from the image frame; and infer, using a post-processing model, a side branch mask from the indications of the at least one side branch of the vessel, wherein the image frame is captured using a first imaging modality, and wherein the image frame can be co-registered to one or more other image frames based on the side branch mask, wherein the one or more other modality image frames are captured using a second imaging modality different than the first imaging modality.

With further embodiments of the computer-readable storage device, the first imaging modality is angiography and wherein the second imaging modality is intravascular ultrasound or optical coherence tomography or CT angiography.

With further embodiments of the computer-readable storage device, the instructions when executed further cause the apparatus to co-register the first image frame with the one or more other image frames based in part on the side branch mask.

With further embodiments of the computer-readable storage device, the instructions when executed further cause the apparatus to generate a graphical information element comprising indications of the image frame and the side branch mask and send the graphical information element to a display.

With further embodiments of the computer-readable storage device, the instructions when executed to infer indications of the at least one side branch of the vessel from the image frame cause the apparatus to infer indications of the at least one side branch of the vessel from the image frame and a vessel centerline.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a side branch identification system in accordance with at least one embodiment.

FIG. 2 illustrates an example image frame, vessel centerline, and inferred side branch mask in accordance with at least one embodiment.

FIG. 3 illustrates an example image frame, vessel centerline, intravascular images, and inferred side branch mask in accordance with at least one embodiment.

FIG. 4 illustrates a routine for inferring a side branch mask using an ensemble of ML networks in accordance with at least one embodiment.

FIG. 5 illustrates another routine for inferring a side branch mask using an ensemble of ML networks in accordance with at least one embodiment.

FIG. 6A and FIG. 6B illustrate exemplary artificial intelligence/machine learning (AI/ML) systems suitable for use with at least one embodiments.

FIG. 7 illustrates a computer-readable storage medium in accordance with at least one embodiment.

FIG. 8 illustrates an example imaging system in accordance with at least one embodiment.

FIG. 9 illustrates a diagrammatic representation of a machine in the form of a computer system within which a set of instructions may be executed for causing the machine to perform any one or more of the methodologies discussed herein, according to an example embodiment.

### DETAILED DESCRIPTION

As noted above, the present disclosure provides methods and apparatuses for side branch detection in coronary angiography images. In general, the disclosure provides on ensemble of machine learning (ML) networks arranged to infer the side branches and their locations from an external coronary image (e.g., angiography) or the external coronary image and intravascular coronary images (e.g., IVUS). The ensemble of ML networks includes several initial ML models whose raw predictions are combined and used as input to a post-processing ML model. The output from the post-processing model can comprise indication of the side branch locations on the coronary angiography image. With some embodiments, the present disclosure can include yet another ML model trained to “pair” or match the detected side branches from different imaging modalities with each other.

As noted, side branches play a key role for co-registration of different coronary artery image modalities. Accurate detection of side branch locations on both image modalities is crucial for a successful co-registration. Conventionally, side branch detection requires input from the user or multiple external images. The present disclosure provides a significant advantage and improvement to the technology of co-registration as the present disclosure provide for identification of side branches from just a single external image and/or with limited or no user input. The present disclosure can be implemented as part of a co-registration technique to improve the alignment between an angiographic image and a series of intravascular images.

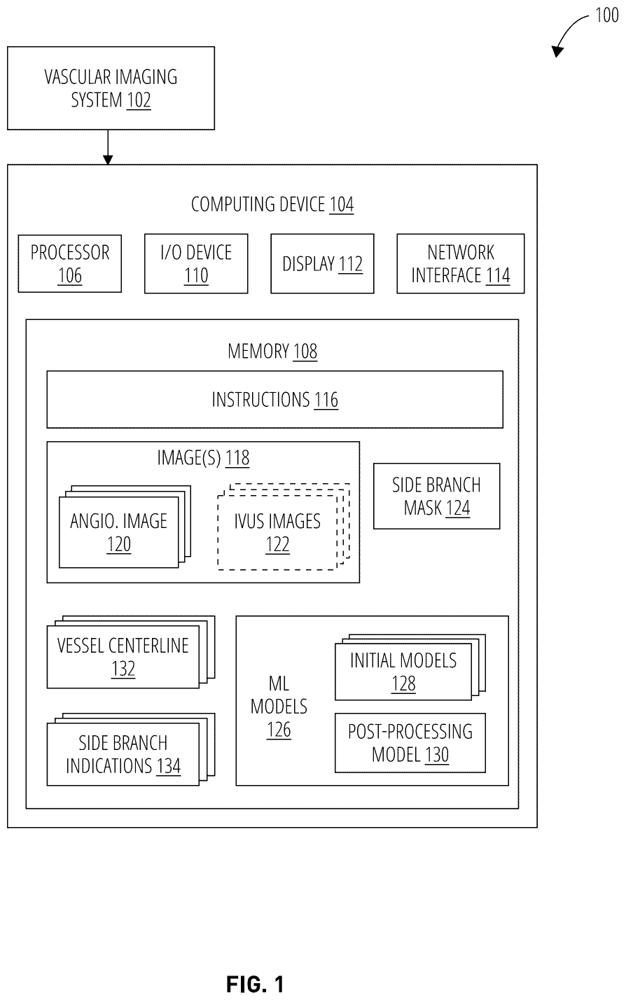

FIG. 1 illustrates a side branch detection system 100, in accordance with an embodiment of the present disclosure. In general, side branch detection system 100 is a system configured to identify side branches from an extravascular (e.g., angiographic, or the like) image of a vessel. In further embodiments, side branch detection system 100 can be configured to identify side branches from an extravascular image and intravascular images. The side branch detection system 100 is configured to receive image(s) 118 which can include angiography image 120 or angiography image 120 and IVUS images 122 and to identify a side branch mask 124 indicating locations of side branches on the angiography image 120.

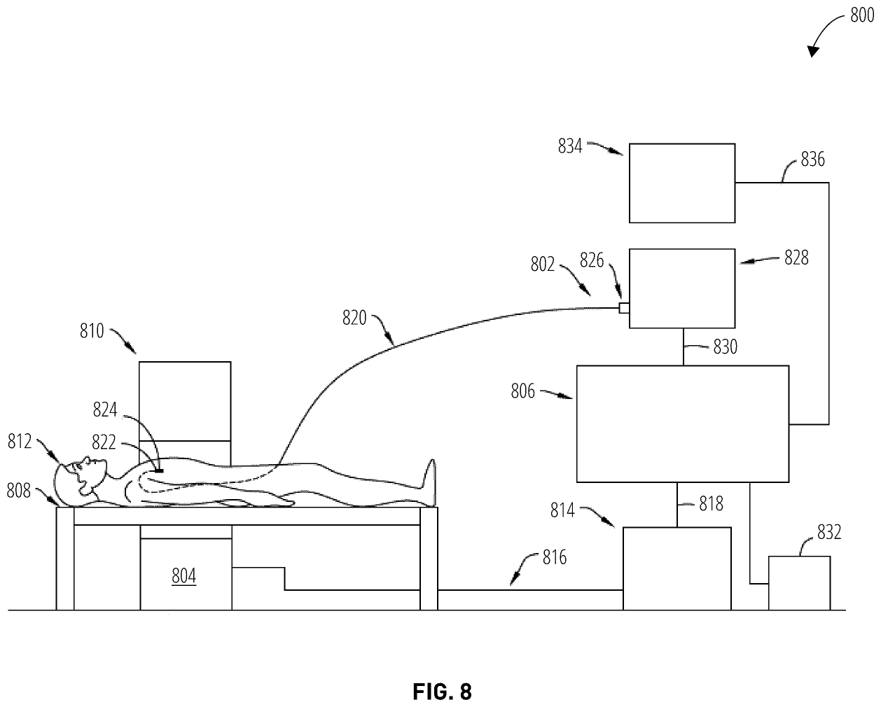

To that end, side branch detection system 100 includes, or can be coupled to, vascular imaging system 102. Vascular imaging system 102 can be any of a variety of vascular imagers (e.g., external, internal, both external and internal, or the like). An example of a vascular imager configured to capture both external and internal vascular images (e.g., angiography image 120 and IVUS images 122) is described with reference to the combined internal and external imaging system 800 depicted in FIG. 8.

Side branch detection system 100 includes computing device 104. Computing device 104 can be any of a variety of computing devices. In some embodiments, computing device 104 can be incorporated into and/or implemented by a console of vascular imaging system 102. With some embodiments, computing device 104 can be a tablet, laptop, workstation, or server communicatively coupled to vascular imaging system 102. With still other embodiments, computing device 104 can be provided by a cloud based computing device, such as, by a Computing as a Service (CaaS) system accessibly over a network (e.g., the Internet, an intranet, a wide area network, or the like). Computing device 104 can include processor 106, memory 108, input and/or output (I/O) device 110, and network interface 114.

The processor 106 may include circuity or processor logic, such as, for example, any of a variety of commercial processors. In some examples, processor 106 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 106 may include graphics processing portions and may include dedicated memory, multiple-threaded processing and/or some other parallel processing capability. In some examples, the processor 106 may be an application specific integrated circuit (ASIC) or a field programmable integrated circuit (FPGA).

The memory 108 may include logic, a portion of which includes arrays of integrated circuits, forming non-volatile memory to persistently store data or a combination of non-volatile memory and volatile memory. It is to be appreciated, that the memory 108 may be based on any of a variety of technologies. In particular, the arrays of integrated circuits included in memory 108 may be arranged to form one or more types of memory, such as, for example, dynamic random access memory (DRAM), NAND memory, NOR memory, or the like.

I/O devices 110 can be any of a variety of devices to receive input and/or provide output. For example, I/O devices 110 can include, a keyboard, a mouse, a joystick, a foot pedal, a haptic feedback device, an LED, or the like. Display 112 can be a conventional display or a touch-enabled display. Further, display 112 can utilize a variety of display technologies, such as, liquid crystal display (LCD), light emitting diode (LED), or organic light emitting diode (OLED), or the like.

Network interface 114 can include logic and/or features to support a communication interface. For example, network interface 114 may include one or more interfaces that operate according to various communication protocols or standards to communicate over direct or network communication links. Direct communications may occur via use of communication protocols or standards described in one or more industry standards (including progenies and variants). For example, network interface 114 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 114 can include logic and/or features to enable communication over a variety of wired or wireless network standards (e.g., 802.11 communication standards). For example, network interface 114 may be arranged to support wired communication protocols or standards, such as, Ethernet, or the like. As another example, network interface 114 may be arranged to support wireless communication protocols or standards, such as, for example, Wi-Fi, Bluetooth, ZigBee, LTE, 5G, or the like.

Memory 108 can include instructions 116, image(s) 118, side branch mask 124, ML models 126, vessel centerline 132, and side branch indications 134. As introduced above, image(s) 118 can include angiography image 120 or both angiography image 120 and IVUS images 122. ML models 126 can include initial models 128 and post-processing model 130. It is noted that initial models 128 includes multiple ML models while post-processing model 130 includes one ML model.

During operation, processor 106 can execute instructions 116 to cause computing device 104 to receive image(s) 118 from vascular imaging system 102. Image(s) 118 includes angiography image 120, which can be a CT images of a patient's heart or portion of a patient heart captured after injection of a contrast agent into the patient's vasculature. Further, image(s) 118 can optionally include IVUS images 122, which can be ultrasound images captured by an ultrasound transducer as it is moved through a portion of the vessel. Other extravascular and intravascular image modalities can be implemented. However, the present disclosure references angiography and IVUS for case of reference.

Processor 106 can further execute instructions 116 to cause computing device 104 to infer side branch mask 124 from image(s) 118 using ML models 126. In, processor 106 can execute instructions 116 to infer side branch indications 134 from image(s) 118 using initial models 128 and to infer side branch mask 124 from side branch indications 134 using post-processing model 130.

In some examples, angiography image 120 can be a single image frame while in other examples, angiography image 120 can be multiple image frames in a series of angiography images (e.g., cine loop, or the like). IVUS images 122 can include multiple image frames. In the case where angiography image 120 is multiple image frames, side branch mask 124 can correspond to or be defined with respect to a one of the frames (e.g., the frame with the highest contrast, or the like).

With some embodiments, initial models 128 can be configured to receive image(s) 118 and one or more indications of the vessel structure, such as, vessel centerline 132, segmentation masks of angiography image 120, or the like. In such examples, processor 106 can execute instructions 116 to cause computing device 104 to determine the vessel indications (e.g., vessel centerline 132, segmentation masks, or the like). In other examples, processor 106 can execute instructions 116 to cause computing device 104 to receive (e.g., via network interfaces 114, or the like) or retrieve (e.g., from memory 108, or the like) the vessel indications.

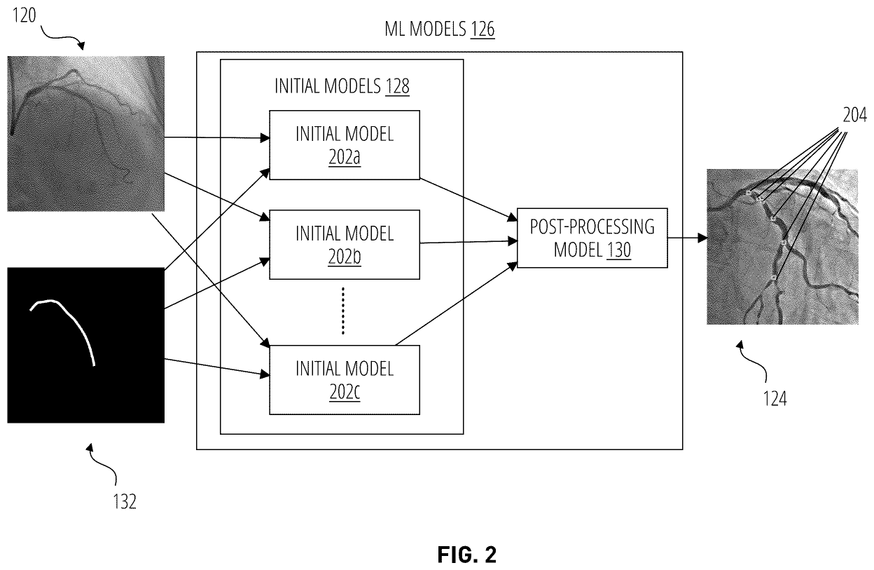

FIG. 2 depicts an example of a side branch mask 124, which can be inferred from ML models 126. For example, initial models 128 may include any number of ML models. This figure depicts initial models 128 including three (3) ML models, initial models 202a, 202b, and 202c. However, in practice initial models 128 can include any integer number of ML models greater two (2). Initial models 128 can infer an output (e.g., side branch indications 134, or the like) which is not shown in this figure for purposes of clarity from angiography image 120. In some examples, initial models 128 can be configured to infer side branch indications 134 from angiography image 120 and vessel centerline 132. In some examples, angiography image 120 and vessel centerline 132 can be combined (e.g., vessel centerline 132 can be overlaid on angiography image 120, or the like) while in other embodiments, angiography image 120 and vessel centerline 132 can be distinct data structures. With some examples, angiography image 120 is a 256 pixel by 256 pixel angiography image. With some examples, initial models 128 can infer side branch indications 134 from angiography image 120 and at least one vessel indications (e.g., vessel centerline 132, a segmentation mask, or the like).

In general, side branch indications 134 can be any ML model output (e.g., scores and corresponding 1D locations along vessel centerline 132, a coordinate (or coordinates) on angiography image 120, or the like.

The output from initial models 128 (e.g., side branch indications 134, or the like) can be used as input to post-processing model 130, which itself can be configured to infer side branch mask 124. Side branch mask 124 can be a one-dimensional (1D) location (or locations) of a side branch (or branches) defined with respect to angiography image 120. In some embodiments, side branch mask 124 can be a copy of the angiography image 120 with side branch locations 204 overlaid onto the image (e.g., as shown). With other embodiments, side branch mask 124 can be a vector of pixel locations defined with respect to the vessel centerline 132 overlaid onto the angiography image 120. With still other embodiments, side branch mask 124 can be a matrix of pixel locations.

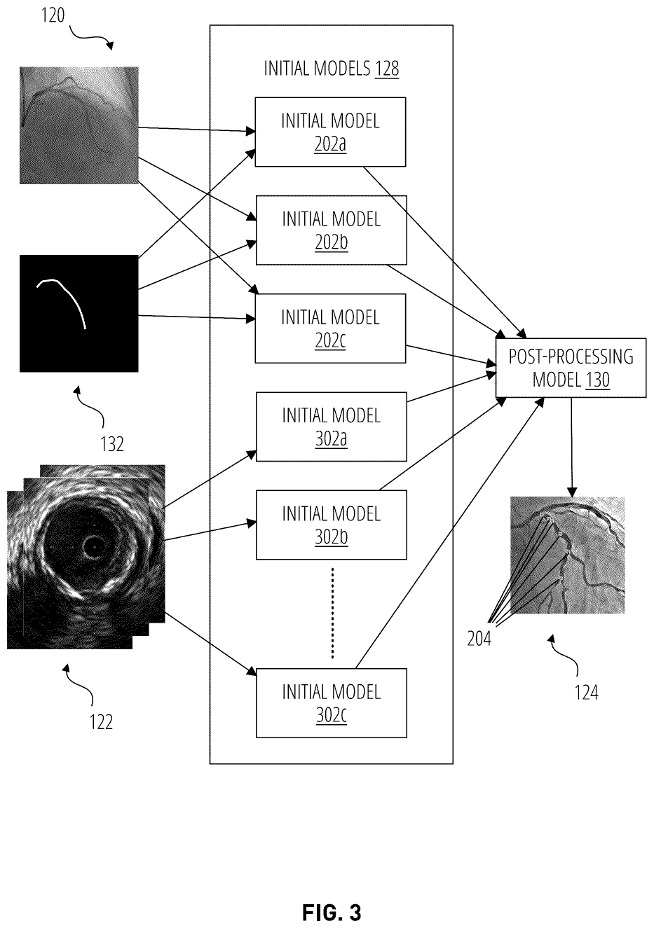

FIG. 3 depicts another example of a side branch mask 124 detailing side branch locations 204, which can be inferred from ML models 126. For example, FIG. 3 depicts initial models 128 including the three (3) initial models 202a, 202b, and 202c and three (3) more initial models 128, which are initial models 302a, 302b, and 302c. The initial models 302a, 302b, and 302c can be configured to infer an output (e.g., side branch indications 134,) also not shown in this figure for clarity, from IVUS images 122. It is noted that although initial models 128 depicts three (3) ML models configured to infer side branch indications 134 from angiography image 120 and three (3) other ML models configured to infer side branch indications 134 from IVUS images 122, these numbers need not be the same. For example, where initial models 128 includes models configured to infer side branch indications 134 from both angiography image 120 and IVUS images 122, a subset of the initial models 128 can be configured to infer a subset of side branch indications 134 from angiography image 120 while another subset of initial models 128 can be configured to infer another subset of side branch indications 134 from IVUS images 122 where the number of ML models in each subset need not be the same.

Further, contrary to that depicted in this figure, one or more of the ML models of initial models 128 can be configured to receive both angiography image 120 and IVUS images 122 as input and infer side branch indications 134 from the input.

Like in FIG. 2, the network topology in FIG. 3 can be arranged such that the output from initial models 128 (e.g., side branch indications 134, or the like) can be used as input to post-processing model 130, which itself can be configured to infer side branch mask 124.

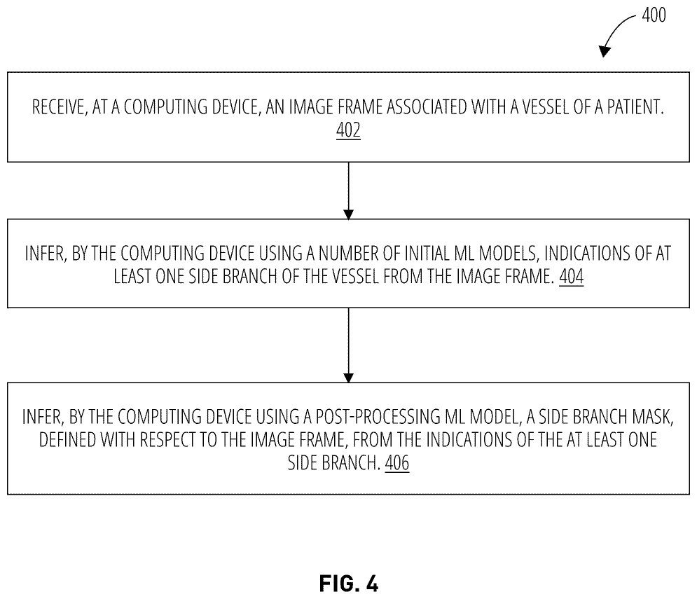

FIG. 4 illustrates routines 400 and 500 respectively, according to some embodiments of the present disclosure. Routines 400 and 500 can be implemented by side branch detection system 100, or another computing device, as outlined herein to identify side branches of a vessel represented in an angiography image (or series of images). Routines 400 and 500 can be implemented to generate indications of locations of side branches defined with respect to an external image of a vessel. For example, routine 400 can be configured to infer side branch mask 124 from angiography image 120 while routine 500 can be configured to infer side branch mask 124 from angiography image 120 and IVUS images 122.

It is noted that routines 400 and 500 are described with reference to a single angiography image. However, routines 400 and 500 could be repeated iteratively on multiple angiography images. As another example, each block or step of routines 400 and 500 could be performed on multiple angiography images.

Routine 400 can begin at block 402 “receive, at a computing device, an image frame associated with a vessel of a patient” an angiography image frame can be received at a computing device. For example, computing device 104 of side branch detection system 100 can receive angiography image 120. With some embodiments, angiography image 120 can be received from vascular imaging system 102 while in other embodiments angiography image 120 can have been previously captured by vascular imaging system 102 and stored in memory (e.g., memory 108, a memory location accessible over network interface 114). In such examples, computing device 104 can access angiography image 120 from the memory location. With some embodiments, processor 106 can execute instructions 116 to receive an indication from a user of a frame of several angiography image frames (e.g., a cine-loop, or the like) to use as angiography image 120. In other embodiments, processor 106 can execute instructions 116 to identify a frame of several angiography image frames (e.g., a cine-loop, or the like) to use as angiography image 120 based on the one of the frames having the highest contrast, the most segmentation, or the like.

Continuing to block 404 “infer, by the computing device using a number of initial ML models, indications of at least one side branch of the vessel from the image frame” indications of at least one side branch of the vessel can be inferred from the image frame using several initial ML models. For example, processor 106 can execute instructions 116 to infer side branch indications 134 from angiography image 120 using initial models 128.

Continuing to block 406 “infer, by the computing device using a post-processing ML model, a side branch mask, defined with respect to the image frame, from the indications of the at least one side branch” a side branch mask, defined with respect to the image frame, can be inferred from the indications of the at least one side branch using a post-processing ML model. For example, processor 106 can execute instructions 116 to infer side branch mask 124 from side branch indications 134 using post-processing model 130.

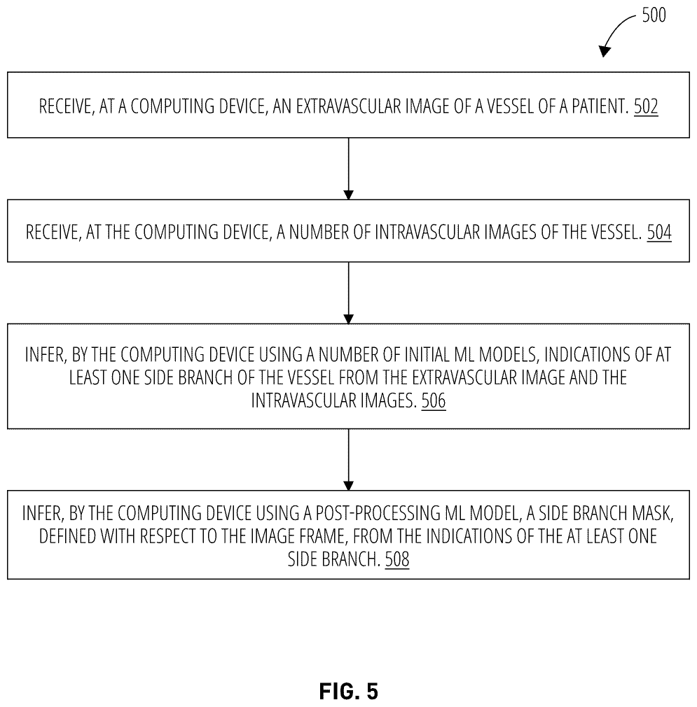

FIG. 5 illustrates routines 500, which can begin at block 502. At block 502 “receive, at a computing device, an extravascular image of a vessel of a patient” an angiography image frame can be received at a computing device. For example, computing device 104 of side branch detection system 100 can receive angiography image 120. With some embodiments, angiography image 120 can be received from vascular imaging system 102 while in other embodiments angiography image 120 can have been previously captured by vascular imaging system 102 and stored in memory (e.g., memory 108, a memory location accessible over network interface 114). In such examples, computing device 104 can access angiography image 120 from the memory location. With some embodiments, processor 106 can execute instructions 116 to receive an indication from a user of a frame of several angiography image frames (e.g., a cine-loop, or the like) to use as angiography image 120. In other embodiments, processor 106 can execute instructions 116 to identify a frame of several angiography image frames (e.g., a cine-loop, or the like) to use as angiography image 120 based on the one of the frames having the highest contrast, the most segmentation, or the like.

Continuing to block 504 “receive, at the computing device, a number of intravascular images of the vessel” intravascular image frames can be received at the computing device. For example, computing device 104 of side branch detection system 100 can receive IVUS images 122. With some embodiments, IVUS images 122 can be received from vascular imaging system 102 while in other embodiments IVUS images 122 can have been previously captured by vascular imaging system 102 and stored in memory (e.g., memory 108, a memory location accessible over network interface 114). In such examples, computing device 104 can access IVUS images 122 from the memory location.

Continuing to block 506 “infer, by the computing device using a number of initial ML models, indications of at least one side branch of the vessel from the extravascular image and the intravascular images” indications of at least one side branch of the vessel can be inferred from the extravascular image and the intravascular images using several initial ML models. For example, processor 106 can execute instructions 116 to infer side branch indications 134 from angiography image 120 and IVUS images 122 using initial models 128.

Continuing to block 508 “infer, by the computing device using a post-processing ML model, a side branch mask, defined with respect to the image frame, from the indications of the at least one side branch” a side branch mask, defined with respect to the image frame, can be inferred from the indications of the at least one side branch using a post-processing ML model. For example, processor 106 can execute instructions 116 to infer side branch mask 124 from side branch indications 134 using post-processing model 130.

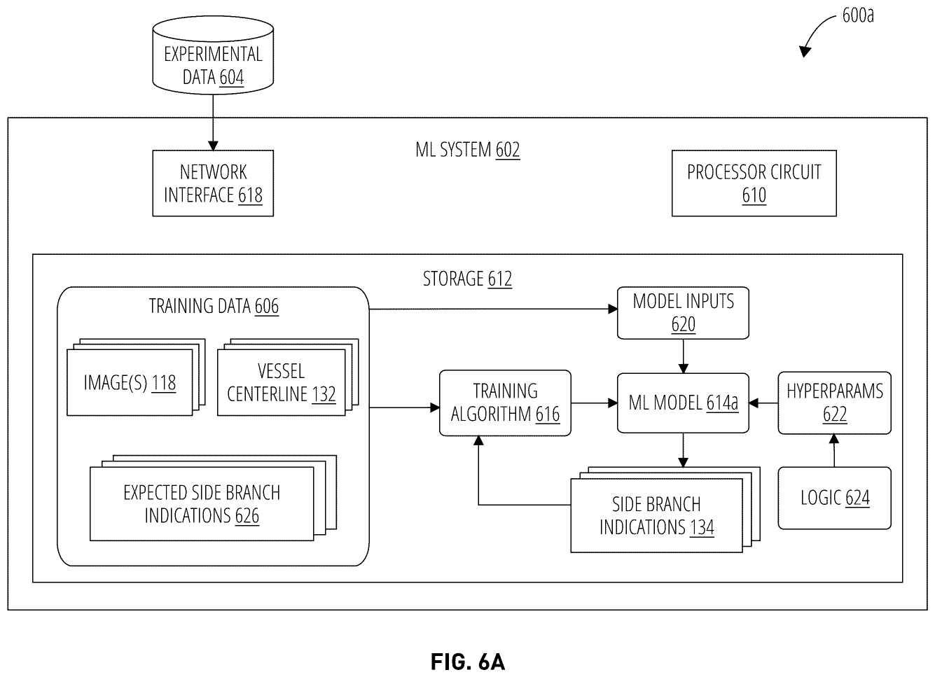

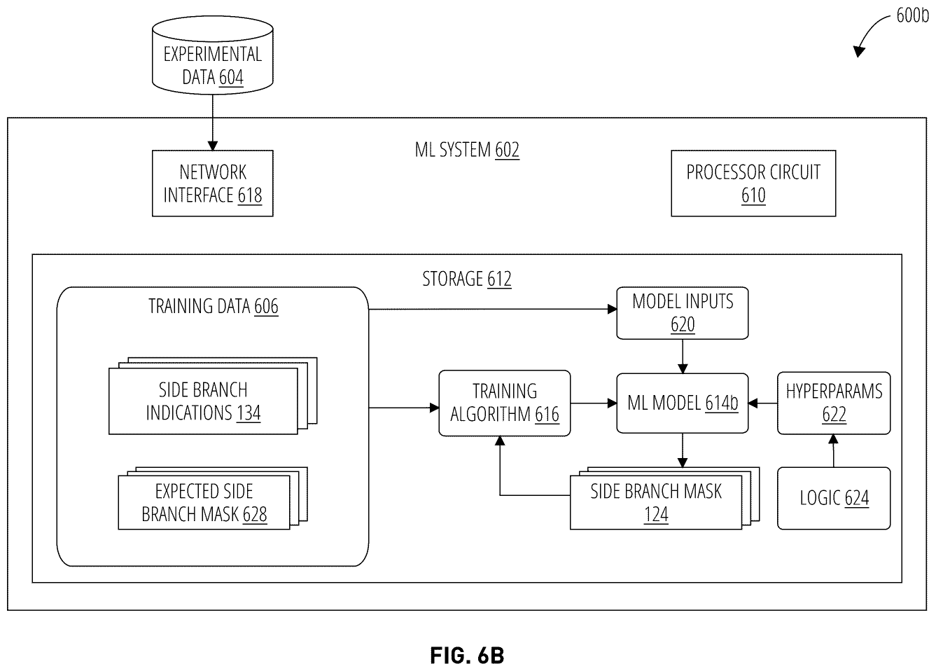

As noted, with some embodiments, an ML model can be utilized to infer side branch locations, or a side branch mask, where the side branch mask is defined with respect to an extravascular image of a vessel. For example, processor 106 of computing device 104 can execute instructions 116 to infer side branch mask 124 from image(s) 118 using ML models 126 (e.g., initial models 128 and post-processing model 130). In such examples, the ML models (e.g., ML models 126) can be stored in memory 108 of computing device 104. It will be appreciated however, that prior to being deployed, the ML models are to be trained. FIG. 6A illustrates ML training environment 600a, which can be used to train an ML model that may later be used to generate (or infer) side branch indications 134 from image(s) 118 as described herein. The ML training environment 600a may include an ML System 602, such as a computing device that applies an ML algorithm to learn relationships. In this example, the ML algorithm can learn relationships between a set of inputs (e.g., image(s) 118 and vessel centerline 132) and an output (e.g., side branch indications 134).

The ML System 602 may make use of experimental data 604 gathered during several prior procedures. Experimental data 604 can include image(s) 118 for several patients. The experimental data 604 may be collocated with the ML System 602 (e.g., stored in a storage 612 of the ML System 602), may be remote from the ML System 602 and accessed via a network interface 618, or may be a combination of local and remote data.

Experimental data 604 can be used to form training data 606, which includes the image(s) 118 (e.g., angiography image 120, IVUS images 122, etc.) and vessel centerline 132.

As noted above, the ML System 602 may include a storage 612, which may include a hard drive, solid state storage, and/or random access memory. The storage 612 may hold training data 606. In general, training data 606 can include information elements or data structures comprising indications of image(s) 118 and associated expected side branch indications 626. The training data 606 may be applied to train an ML model 614a. Depending on the application, different types of models may be used to form the basis of ML model 614a. For instance, in the present example, an artificial neural network (ANN) may be particularly well-suited to learning associations between CT angiography images and/or IVUS images (e.g., image(s) 118, or the like) and side branch indications 134 (e.g., indications of locations of one or more side branches on angiography image 120. Convoluted neural networks may also be well-suited to this task. Any suitable training algorithm 616 may be used to train the ML model 614a. Nonetheless, the example depicted in FIG. 6A may be particularly well-suited to a supervised training algorithm or reinforcement learning training algorithm. For a supervised training algorithm, the ML System 602 may apply the image(s) 118 as model inputs 620, to which expected side branch indications 626 may be mapped to learn associations between the image(s) 118 and the side branch indications 134. In a reinforcement learning scenario, training algorithm 616 may attempt to maximize some or all (or a weighted combination) of the model inputs 620 mappings to side branch indications 134 to produce ML model 614a having the least error. With some embodiments, training data 606 can be split into “training” and “testing” data wherein some subset of the training data 606 can be used to adjust the ML model 614a (e.g., internal weights of the model, or the like) while another, non-overlapping subset of the training data 606 can be used to measure an accuracy of the ML model 614a to infer (or generalize) IVUS images 122 from “unseen” training data 606 (e.g., training data 606 not used to train ML model 614a).

The ML model 614a may be applied using a processor circuit 610, which may include suitable hardware processing resources that operate on the logic and structures in the storage 612. The training algorithm 616 and/or the development of the trained ML model 614a may be at least partially dependent on hyperparameters 622. In exemplary embodiments, the model hyperparameters 622 may be automatically selected based on hyperparameter optimization logic 624, which may include any known hyperparameter optimization techniques as appropriate to the ML model 614a selected and the training algorithm 616 to be used. In optional, embodiments, the ML model 614a may be re-trained over time, to accommodate new knowledge and/or updated experimental data 604.

Once the ML model 614a is trained, it may be applied (e.g., by the processor circuit 610, by processor 106, or the like) to new input data (e.g., image(s) 118 captured during a pre-PCI intervention, a post-PCI intervention, or the like). This input to the ML model 614a may be formatted according to a predefined model inputs 620 mirroring the way that the training data 606 was provided to the ML model 614a. Trained model ML model 614a may generate side branch indications 134 from image(s) 118. In such examples, ML model 614a can be deployed as one of initial models 128. It is noted that in the present disclosure, initial models 128 include multiple ML models (e.g., initial models 202a, 202b, 202c, 302a, 302b, 302c, etc.) As such, multiple ML model 614a can be trained using the process outlined above to produce several initial models 128.

ML System 602 can further be utilized to train a model to infer side branch mask 124 from side branch indications 134. FIG. 6B illustrates ML training environment 600b, which is an example of ML training environment 600a configured to train ML model 614b to infer side branch mask 124 from side branch indications 134. As such, training data 606 can include side branch indications 134 and expected side branch mask 628 while ML model 614b can be “trained” as outlined above to infer side branch mask 124 from side branch indications 134. Trained model ML model 614b may generate side branch mask 124 from side branch indications 134. In such examples, ML model 614b can be deployed as post-processing model 130.

The above descriptions pertain to a particular kind of ML System 602, which applies supervised learning techniques given available training data with input/result pairs. However, the present invention is not limited to use with a specific ML paradigm, and other types of ML techniques may be used. For example, in some embodiments the ML System 602 may apply for example, evolutionary algorithms, or other types of ML algorithms and models to generate side branch indications 134 or side branch mask 124 as described.

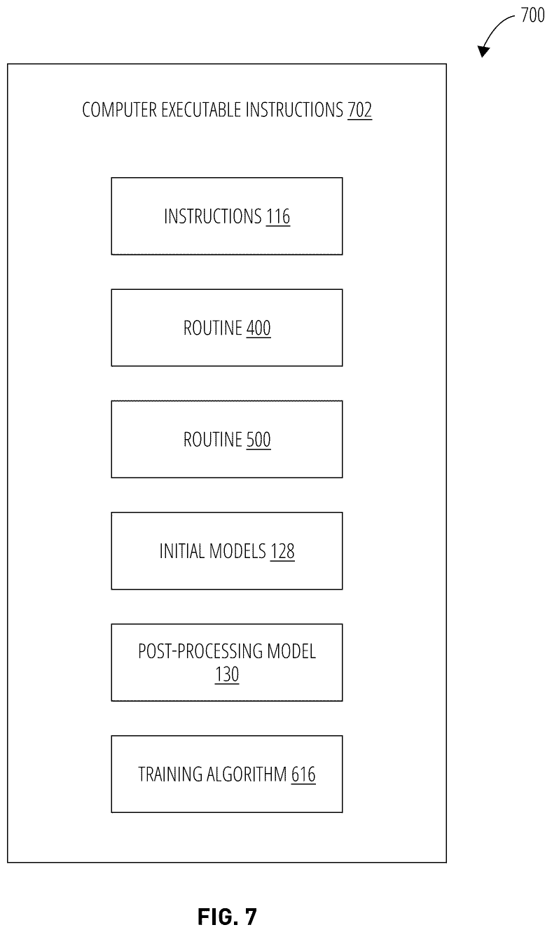

FIG. 7 illustrates computer-readable storage medium 700. Computer-readable storage medium 700 may comprise any non-transitory computer-readable storage medium or machine-readable storage medium, such as an optical, magnetic or semiconductor storage medium. In various embodiments, computer-readable storage medium 700 may comprise an article of manufacture. In some embodiments, computer-readable storage medium 700 may store computer executable instructions 702 with which circuitry (e.g., processor 106, or the like) can execute. For example, computer executable instructions 702 can include instructions to implement operations described with respect side branch detection system 100, which can improve the functioning of side branch detection system 100 as detailed herein. For example, computer executable instructions 702 can include instructions that can cause a computing device to implemented routine 400 of FIG. 4, routine 500 of FIG. 5, training algorithm 616 of FIG. 6A and FIG. 6B. As another example, computer executable instructions 702 can include instructions 116, initial models 128, and/or post-processing model 130. Examples of computer-readable storage medium 700 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 702 may include any suitable type of code, such as source code, compiled code, interpreted code, executable code, static code, dynamic code, object-oriented code, visual code, and the like.

FIG. 8 illustrates a combined internal and external imaging system 800 including both an endoluminal imaging system 802 (e.g., an IVUS imaging system, or the like) and an extravascular imaging system 804 (e.g., an angiographic imaging system). Combined internal and external imaging system 800 further includes computing device 806, which includes circuitry, controllers, and/or processor(s) and memory and software as needed. With some embodiments, side branch detection system 100 can be incorporated into computing device 806 or side branch detection system 100 can incorporate computing device 806. In general, the endoluminal imaging system 802 can be arranged to generate intravascular imaging data (e.g., IVUS images, or the like) while the extravascular imaging system 804 can be arranged to generate extravascular imaging data (e.g., angiography images, or the like).

It is to be appreciated that although the systems and methods described herein do not need endoluminal images, a combined imaging system is described for clarity of presentation. For example, the side branch identification techniques described herein can be implemented with just one extravascular image (or multiple extravascular images). However, as detailed above, the side branch identification techniques can be implemented with just one extravascular images and intravascular images.

The extravascular imaging system 804 may include a table 808 that may be arranged to provide sufficient space for the positioning of an angiography/fluoroscopy unit c-arm 810 in an operative position in relation to a patient 812 on the drive unit. C-arm 810 can be configured to acquires fluoroscopic images in the absence of contrast agent in the blood vessels of the patient 812 and/or acquire angiographic image while there is a presence of contrast agent in the blood vessels of the patient 812.

Raw radiological image data acquired by the c-arm 810 may be passed to an extravascular data input port 814 via a transmission cable 816. The input port 814 may be a separate component or may be integrated into or be part of the computing device 806. The input port 814 may include a processor that converts the raw radiological image data received thereby into extravascular image data (e.g., angiographic/fluoroscopic image data), for example, in the form of live video, DICOM, or a series of individual images. The extravascular image data may be initially stored in memory within the input port 814 or may be stored within memory of computing device 806. If the input port 814 is a separate component from the computing device 806, the extravascular image data may be transferred to the computing device 806 through the transmission cable 816 and into an input port (not shown) of the computing device 806. In some alternatives, the communications between the devices or processors may be carried out via wireless communication, rather than by cables as depicted.

The intravascular imaging data may be, for example, IVUS data or OCT data obtained by the endoluminal imaging system 802. The endoluminal imaging system 802 may include an intravascular imaging device such as an imaging catheter 820. The imaging catheter 820 is configured to be inserted within the patient 812 so that its distal end, including a diagnostic assembly or probe 822 (e.g., an IVUS probe), is in the vicinity of a desired imaging location of a blood vessel. A radiopaque material or marker 824 located on or near the probe 822 may provide indicia of a current location of the probe 822 in a radiological image. In some embodiments, imaging catheter 820 and/or probe 822 can include a guide catheter (not shown) that has been inserted into a lumen of the subject (e.g., a blood vessel, such as a coronary artery) over a guidewire (also not shown). However, in some embodiments, the imaging catheter 820 and/or probe 822 can be inserted into the vessel of the patient 812 without a guidewire.

With some embodiments, imaging catheter 820 and/or probe 822 can include both imaging capabilities as well as other data-acquisition capabilities. For example, FFR and/or iFR data, data related to pressure, flow, temperature, electrical activity, oxygenation, biochemical composition, or any combination thereof. In some embodiments, imaging catheter 820 and/or probe 822 can further include a therapeutic device, such as a stent, a balloon (e.g., an angioplasty balloon), a graft, a filter, a valve, and/or a different type of therapeutic endoluminal device.

Imaging catheter 820 is coupled to a proximal connector 826 to couple imaging catheter 820 to image acquisition device 828. Image acquisition device 828 may be coupled to computing device 806 via transmission cable 816, or a wireless connection. The intravascular image data may be initially stored in memory within the image acquisition device 828 or may be stored within memory of computing device 806. If the image acquisition device 828 is a separate component from computing device 806, the intravascular image data may be transferred to the computing device 806, via, for example, transmission cable 816.

The computing device 806 can also include one or more additional output ports for transferring data to other devices. For example, the computer can include an output port to transfer data to a data archive or memory device 832. The computing device 806 can also include a user interface (described in greater detail below) that includes a combination of circuitry, processing components and instructions executable by the processing components and/or circuitry to enable the image identification and vessel routing or pathfinding described herein and/or dynamic co-registration of intravascular and extravascular images using the identified vessel pathway.

In some embodiments, computing device 806 can include user interface devices, such as, a keyboard, a mouse, a joystick, a touchscreen device (such as a smartphone or a tablet computer), a touchpad, a trackball, a voice-command interface, and/or other types of user interfaces that are known in the art.

The user interface can be rendered and displayed on display 834 coupled to computing device 806 via display cable 836. Although the display 834 is depicted as separate from computing device 806, in some examples the display 834 can be part of computing device 806. Alternatively, the display 834 can be remote and wireless from computing device 806. As another example, the display 834 can be part of another computing device different from computing device 806, such as, a tablet computer, which can be coupled to computing device 806 via a wired or wireless connection. For some applications, the display 834 includes a head-up display and/or a head-mounted display. For some applications, the computing device 806 generates an output on a different type of visual, text, graphics, tactile, audio, and/or video output device, e.g., speakers, headphones, a smartphone, or a tablet computer. For some applications, the user interface rendered on display 834 acts as both an input device and an output device.

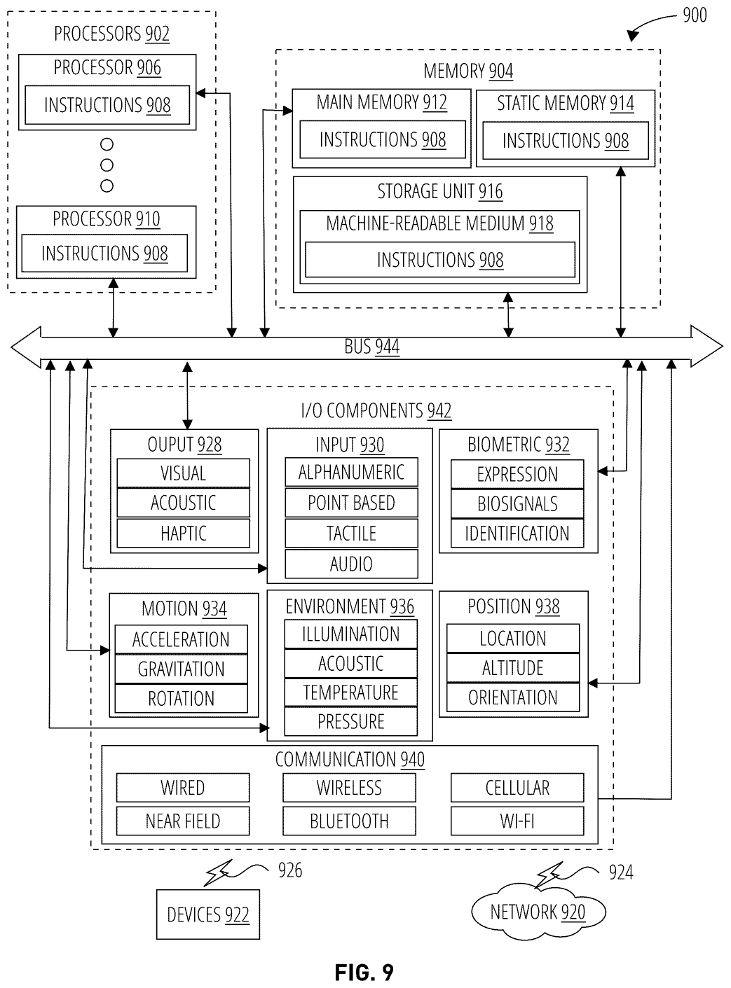

FIG. 9 illustrates a diagrammatic representation of a machine 900 in the form of a computer system within which a set of instructions may be executed for causing the machine to perform any one or more of the methodologies discussed herein. More specifically, FIG. 9 shows a diagrammatic representation of the machine 900 in the example form of a computer system, within which instructions 908 (e.g., software, a program, an application, an applet, an app, or other executable code) for causing the machine 900 to perform any one or more of the methodologies discussed herein may be executed. For example, the instructions 908 may cause the machine 900 to execute instructions 116, routine 400 of FIG. 4, routine 500 of FIG. 5, training algorithm 616 of FIG. 6A or FIG. 6B or the like. More generally, the instructions 908 may cause the machine 900 to identify side branches of a vessel from a CT angiography image or a CT angiography image and intravascular images as described herein.

The instructions 908 transform the general, non-programmed machine 900 into a particular machine 900 programmed to carry out the described and illustrated functions in a specific manner. In alternative embodiments, the machine 900 operates as a standalone device or may be coupled (e.g., networked) to other machines. In a networked deployment, the machine 900 may operate in the capacity of a server machine or a client machine in a server-client network environment, or as a peer machine in a peer-to-peer (or distributed) network environment. The machine 900 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 908, sequentially or otherwise, that specify actions to be taken by the machine 900. Further, while only a single machine 900 is illustrated, the term “machine” shall also be taken to include a collection of machines 200 that individually or jointly execute the instructions 908 to perform any one or more of the methodologies discussed herein.

The machine 900 may include processors 902, memory 904, and I/O components 942, which may be configured to communicate with each other such as via a bus 944. In an example embodiment, the processors 902 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 906 and a processor 910 that may execute the instructions 908. The term “processor” is intended to include multi-core processors that may comprise two or more independent processors (sometimes referred to as “cores”) that may execute instructions contemporaneously. Although FIG. 9 shows multiple processors 902, the machine 900 may include a single processor with a single core, a single processor with multiple cores (e.g., a multi-core processor), multiple processors with a single core, multiple processors with multiples cores, or any combination thereof.

The memory 904 may include a main memory 912, a static memory 914, and a storage unit 916, both accessible to the processors 902 such as via the bus 944. The main memory 904, the static memory 914, and storage unit 916 store the instructions 908 embodying any one or more of the methodologies or functions described herein. The instructions 908 may also reside, completely or partially, within the main memory 912, within the static memory 914, within machine-readable medium 918 within the storage unit 916, within at least one of the processors 902 (e.g., within the processor's cache memory), or any suitable combination thereof, during execution thereof by the machine 900.

The I/O components 942 may include a wide variety of components to receive input, provide output, produce output, transmit information, exchange information, capture measurements, and so on. The specific I/O components 942 that are included in a particular machine will depend on the type of machine. For example, portable machines such as mobile phones will likely include a touch input device or other such input mechanisms, while a headless server machine will likely not include such a touch input device. It will be appreciated that the I/O components 942 may include many other components that are not shown in FIG. 9. The I/O components 942 are grouped according to functionality merely for simplifying the following discussion and the grouping is in no way limiting. In various example embodiments, the I/O components 942 may include output components 928 and input components 930. The output components 928 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 930 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 942 may include biometric components 932, motion components 934, environmental components 936, or position components 938, among a wide array of other components. For example, the biometric components 932 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 934 may include acceleration sensor components (e.g., accelerometer), gravitation sensor components, rotation sensor components (e.g., gyroscope), and so forth. The environmental components 936 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 938 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 942 may include communication components 940 operable to couple the machine 900 to a network 920 or devices 922 via a coupling 924 and a coupling 926, respectively. For example, the communication components 940 may include a network interface component or another suitable device to interface with the network 920. In further examples, the communication components 940 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 922 may be another machine or any of a wide variety of peripheral devices (e.g., a peripheral device coupled via a USB).

Moreover, the communication components 940 may detect identifiers or include components operable to detect identifiers. For example, the communication components 940 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 940, such as location via Internet Protocol (IP) geolocation, location via Wi-Fi® signal triangulation, location via detecting an NFC beacon signal that may indicate a particular location, and so forth.

The various memories (i.e., memory 904, main memory 912, static memory 914, and/or memory of the processors 902) and/or storage unit 916 may store one or more sets of instructions and data structures (e.g., software) embodying or utilized by any one or more of the methodologies or functions described herein. These instructions (e.g., the instructions 908), when executed by processors 902, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 920 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 920 or a portion of the network 920 may include a wireless or cellular network, and the coupling 924 may be a Code Division Multiple Access (CDMA) connection, a Global System for Mobile communications (GSM) connection, or another type of cellular or wireless coupling. In this example, the coupling 924 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 908 may be transmitted or received over the network 920 using a transmission medium via a network interface device (e.g., a network interface component included in the communication components 940) and utilizing any one of several well-known transfer protocols (e.g., hypertext transfer protocol (HTTP)). Similarly, the instructions 908 may be transmitted or received using a transmission medium via the coupling 926 (e.g., a peer-to-peer coupling) to the devices 922. The terms “transmission medium” and “signal medium” mean the same thing and may be used interchangeably in this disclosure. The terms “transmission medium” and “signal medium” shall be taken to include any intangible medium that can store, encoding, or carrying the instructions 908 for execution by the machine 900, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An apparatus for viewing images of a vessel and for identifying side branches from an image, comprising:
a processor and a memory storage device coupled to the processor, the memory comprising instructions executable by the processor, the instructions when executed cause the apparatus to:
receive an image frame associated with a vessel of a patient;
infer, using a plurality of initial machine learning (ML) models, indications of at least one side branch of the vessel from the image frame; and
infer, using a post-processing model, a side branch mask from the indications of the at least one side branch of the vessel,
wherein the image frame is captured using a first imaging modality, and
wherein the image frame can be co-registered to one or more other image frames based on the side branch mask, wherein the one or more other modality image frames are captured using a second imaging modality different than the first imaging modality.

2. The apparatus of claim 1, wherein the first imaging modality is angiography and wherein the second imaging modality is intravascular ultrasound or optical coherence tomography or CT angiography.

3. The apparatus of claim 1, the instructions when executed further cause the apparatus to co-register the first image frame with the one or more other image frames based in part on the side branch mask.

4. The apparatus of claim 1, the instructions when executed further cause the apparatus to generate a graphical information element comprising indications of the image frame and the side branch mask and send the graphical information element to a display.

5. The apparatus of claim 1, the instructions when executed to infer indications of the at least one side branch of the vessel from the image frame cause the apparatus to infer indications of the at least one side branch of the vessel from the image frame and a vessel centerline.

6. The apparatus of claim 5, the instructions when executed further cause the apparatus to receive the vessel centerline.

7. The apparatus of claim 5, the instructions when executed further cause the apparatus to determine the vessel centerline.

8. The apparatus of claim 5, wherein the side branch mask comprises an indication of locations of the one or more side branches with respect to the centerline of the vessel.

9. The apparatus of claim 1, the instructions when executed to infer indications of the at least one side branch of the vessel from the image frame cause the apparatus to infer indications of the at least one side branch of the vessel from the image frame and a segmentation of the image frame.

10. The apparatus of claim 9, the instructions when executed further cause the apparatus to receive the segmentation of the image frame or determine the segmentation of the image frame.

11. The apparatus of claim 1, the instructions when executed further cause the apparatus to:
receive a plurality of intravascular images of the vessel; and
infer indications of the at least one side branch of the vessel from the image frame and the plurality of intravascular image frames.

12. The apparatus of claim 11, the plurality of initial ML models comprising a first group ML models and a second group of ML models, wherein the first group of ML models is different than the second group of ML models, and the instructions when executed to infer indications of the at least one side branch of the vessel from the image frame and the plurality of intravascular image frames cause the apparatus to:
infer, using the first group of ML models, ones of the indications of the at least one side branch of the vessel from the image frame; and
infer, using the second group of ML models, other ones of the indications of the at least one side branch of the vessel from the plurality of intravascular images.

13. The apparatus of claim 1, wherein the side branch mask is a one-dimensional (1D) mask defined with respect to the image frame, wherein the image frame is one of a plurality of image frames in a cine loop, and/or wherein the image frame is a 256 pixel by 256 pixel angiography image.

14. A computer-readable storage device, comprising instructions executable by a processor of a computing device coupled to an intravascular imaging device and/or a fluoroscope device, wherein when executed the instructions cause the computing device to:
receive an image frame associated with a vessel of a patient;
infer, using a plurality of initial machine learning (ML) models, indications of at least one side branch of the vessel from the image frame; and
infer, using a post-processing model, a side branch mask from the indications of the at least one side branch of the vessel,
wherein the image frame is captured using a first imaging modality, and
wherein the image frame can be co-registered to one or more other image frames based on the side branch mask, wherein the one or more other modality image frames are captured using a second imaging modality different than the first imaging modality.

15. The computer-readable storage device of claim 14, wherein the first imaging modality is angiography and wherein the second imaging modality is intravascular ultrasound or optical coherence tomography or CT angiography.

16. The computer-readable storage device of claim 14, the instructions when executed further cause the apparatus to co-register the first image frame with the one or more other image frames based in part on the side branch mask.

17. The computer-readable storage device of claim 14, the instructions when executed further cause the apparatus to generate a graphical information element comprising indications of the image frame and the side branch mask and send the graphical information element to a display.

18. The computer-readable storage device of claim 14, the instructions when executed to infer indications of the at least one side branch of the vessel from the image frame cause the apparatus to infer indications of the at least one side branch of the vessel from the image frame and a vessel centerline.

19. A method for a vascular co-registration system, comprising:
receiving, at a processor of a vascular co-registration system, an image frame associated with a vessel of a patient;
inferring, by the processor using a plurality of initial machine learning (ML) models, indications of at least one side branch of the vessel from the image frame; and
inferring, by the processor using a post-processing model, a side branch mask from the indications of the at least one side branch of the vessel,
wherein the image frame is captured using a first imaging modality, and
wherein the image frame can be co-registered to one or more other image frames based on the side branch mask, wherein the one or more other modality image frames are captured using a second imaging modality different than the first imaging modality.

20. The method of claim 19, wherein the first imaging modality is angiography and wherein the second imaging modality is intravascular ultrasound or optical coherence tomography or CT angiography.
